(19) United States
(12) Patent Application Publication
(10) Pub. No.: US 2024/0229006 A1
(43) Pub. Date: Jul. 11, 2024

(54) Glutamate-Cysteine Ligase Variant and Method of Producing Glutathione Using the Same

(71) Applicant: CJ CHEILJEDANG CORPORATION, Seoul (KR)

(72) Inventors: Eun Bin Yang, Seoul (KR); Cheol Woong Ha, Seoul (KR); Yeonsoo Kim, Seoul (KR); Yeong Eun Im, Seoul (KR)

(73) Assignee: CJ CHEILJEDANG CORPORATION, Seoul (KR)

(21) Appl. No.: 17/914,306

(22) PCT Filed: Mar. 24, 2021
(86) PCT No.: PCT/KR2021/003617

§ 371 Date: Sep. 23, 2022

(30) Foreign Application Priority Data
Mar. 25, 2020 (KR) 10-2020-0036456

(51) Int. Cl. C12N 9/00; C12N 15/81; C12P 21/02

(52) U.S. Cl. CPC C12N 9/93; C12N 15/81; C12P 21/02; C12Y 603/02002

(57) ABSTRACT

Provided are a novel glutamate-cysteine ligase variant and a method of producing glutathione using the same.

### TECHNICAL FIELD

The present disclosure relates a novel glutamate-cysteine ligase variant and a method producing glutathione using the same.

### BACKGROUND ART

As an organic sulfur compound commonly present in most cells, glutathione (GSH) is a tripeptide composed of three amino acids: glycine, glutamate, and cysteine.

Glutathione is present in the living body in a reduced form of glutathione (GSH) and in an oxidized form of glutathione (GSSG). The reduced form of glutathione (GSH), present in a relatively high proportion under normal circumstances, is mainly distributed in the liver and skin cells in the human body and has important roles of an antioxidant function in decomposing and removing reactive oxygen, a detoxification function in removing xenobiotic compounds such as toxic substances, and a whitening function in inhibiting melanin production.

Since glutathione production gradually decreases as the aging process progresses, and a decrease in production of glutathione, which has important roles in antioxidant and detoxification functions, promotes accumulation of reactive oxygen, which is a main cause of aging, there is a need to supply glutathione from the outside (Sipes I G et al., “The role of glutathione in the toxicity of xenobiotic compounds: metabolic activation of 1,2-dibromoethane by glutathione”, Adv Exp Med Biol. 1986; 197:457-67).

Glutathione, which has various functions as described above, has drawn attention as a substance in various fields such as pharmaceuticals, health functional foods, and cosmetics, and is also used to manufacture taste ingredients and food and feed additives. It is known that glutathione has great effects on enriching the taste of a raw ingredient and maintaining rich flavors and may be used, alone or in combination with other substances, as a kokumi flavor enhancer. In general, kokumi substances are known to have richer flavors than umami substances, such as known nucleic acids and monosodium glutamate (MSG), and to be generated by protein decomposition during ripening.

Although there is a growing demand for glutathione for application in various fields, the market therefor is not activated due to high costs for industrial production of glutathione since processes of synthesizing enzymes therefor have not been commercialized due to high production costs, and yields of methods of culturing microorganisms and extracting glutathione therefrom are low.

### DISCLOSURE

### Technical Problem

As a result of intensive efforts to solve the above-described problems, the present inventors have developed a novel glutamate-cysteine ligase variant and found a significant increase in the glutathione producing ability of strains into which the novel glutamate-cysteine ligase is introduced, thereby completing the present disclosure.

### Technical Solution

The present disclosure provides a glutamate-cysteine ligase variant in which an amino acid corresponding to the 86th position from the N-terminus of an amino acid sequence of SEQ ID NO: 1 is substituted with a different amino acid.

The present disclosure provides a polynucleotide encoding the variant and a vector including the same.

The present disclosure provides a microorganism producing glutathione by including at least one of: the variant; the polynucleotide encoding the variant; and the vector including the polynucleotide.

The present disclosure provides a method of producing glutathione including culturing the microorganism.

### Advantageous Effects

The novel glutamate-cysteine ligase variant of the present disclosure significantly increases glutathione production and thus may be used in production of glutathione with a high yield. Since yeasts producing glutathione with high yields, dried products, extracts, cultures, and lysates thereof, and produced glutathione have antioxidant, detoxification, and immunity enhancing effects, they may be efficiently used in cosmetic compositions, food compositions, feed compositions, and pharmaceutical compositions and preparation thereof.

### BEST MODE

The present disclosure will be described in detail. Meanwhile, each description and embodiment disclosed in the present disclosure may be applied to herein to different descriptions and embodiments. In other words, all combinations of various components disclosed in the present disclosure are included within the scope of the present disclosure. Furthermore, the scope of the present disclosure should not be limited by the descriptions provided below.

Those skilled in the art will recognize or be able to ascertain, using no more than routine experimentation, many equivalents to specific embodiments of the present disclosure. Such equivalents are intended to be encompassed in the scope of the following claims.

An aspect of the present disclosure provides a glutamate-cysteine ligase variant in which an amino acid corresponding to the 86th position from the N-terminus of an amino acid sequence of SEQ ID NO: 1 is substituted with a different amino acid.

The variant may be a glutamate-cysteine ligase variant including at least one amino acid substitution in the amino acid sequence of SEQ ID NO: 1, wherein the substitution includes a substitution of the amino acid corresponding to the 86th position from the N-terminus of the SEQ ID NO: 1 with a different amino acid.

Specifically, the variant may be a protein variant in which the 86th amino acid from the N-terminus of the amino acid sequence of SEQ ID NO: 1 is substituted with a different amino acid.

The “glutamate-cysteine ligase (GCL)” of the present disclosure is an enzyme also referred to as “glutamate-cysteine linking enzyme” or “gamma-glutamyl cysteine synthetase (GCS)”. The glutamate-cysteine ligase is known to catalyze the following reaction:

L-glutamate+L-cysteine+ATP↔gamma-glutamyl cysteine+ADP+Pᵢ

In addition, the reaction catalyzed by the glutamate-cysteine ligase is known as a first step of glutathione synthesis.

The glutamate-cysteine ligase, as a yeast-derived sequence, may be a protein including an amino acid sequence of SEQ ID NO: 1, without being limited thereto.

In the present disclosure, the amino acid sequence of SEQ ID NO: 1 is an amino acid sequence encoded by gsh1 gene and may also be referred to as “GSH1 protein” or “glutamate-cysteine ligase”. The amino acid sequence constituting the glutamate-cysteine ligase of the present disclosure may be obtained from a known database of the NCBI GenBank. For example, the amino acid sequence may be derived from Saccharomyces cerevisiae, but is not limited thereto, and may include any sequence having the same activity as the amino acid sequence without limitation.

In addition, although the glutamate-cysteine ligase is defined as a protein including the amino acid sequence of SEQ ID NO: 1 in the present disclosure, it does not exclude a mutation that may occur naturally or by addition of a meaningless sequence upstream or downstream of the amino acid sequence of SEQ ID NO: 1 or a naturally occurring mutation or a silent mutation thereof, and it is obvious to those skilled in the art that any proteins having the activity identical or equivalent to the protein including the amino acid sequence of SEQ ID NO: 1 belong to the glutamate-cysteine ligase of the present disclosure.

For example, the glutamate-cysteine ligase of the present disclosure may be a protein including the amino acid sequence of SEQ ID NO: 1 or an amino acid sequence having at least 80%, 85%, 90%, 95%, 96%, 97%, 98%, or 99% homology or identity therewith. Also, it will be obvious that any protein having the amino acid sequence including a deletion, modification, substitution, or addition of one or several amino acids is within the scope of the present disclosure as long as the amino acid sequence retains the above-described homology or identity and an effect equivalent to that of the protein.

In other words, although the expressions “protein or polypeptide having an amino acid sequence of a predetermined SEQ ID NO:” and “protein or polypeptide including an amino acid sequence of a predetermined SEQ ID NO:” are used in the present disclosure, it is obvious that any protein having the amino acid sequence including a deletion, modification, substitution, or addition of one or several amino acids may also be used in the present disclosure, as long as the protein has an activity identical or equivalent to the polypeptide consisting of the predetermined amino acid sequence. For example, it is obvious that a “polypeptide including the amino acid sequence of SEQ ID NO: 1” belongs to a “polypeptide consisting of the amino acid sequence of SEQ ID NO: 1” as long as the former has the same or an equivalent activity to the latter.

As used herein, the term “variant” refers to a protein obtained by conservative substitution and/or modification of at least one amino acid different from that of the recited sequence while retaining functions or properties of proteins, and may be a variant of the glutamate-cysteine ligase in which the amino acid corresponding to the 86^{th} position from the N-terminus of an amino acid sequence of SEQ ID NO: 1 is substituted with an amino acid residue other than cysteine in view of the objects of the present disclosure. The variant is different from the sequence identified by substitution, deletion, or addition of several amino acids. Such variants may generally be identified by modifying one of the above amino acid sequences of the protein and evaluating properties of the modified protein. That is, the ability of the variant may be enhanced relative to a native protein. In addition, some variants may include variants from which at least one portion such as a N-terminus leader sequence or a transmembrane domain have been removed. Other variants may include variants in which a portion has been removed from the N- and/or C-terminus of a mature protein. The term “variant” may also be used interchangeably with other terms such as modification, modified protein, modified polypeptide, mutant, mutein, and divergent, and any terms used to indicate variation may also be used without limitation. In view of the objects of the present disclosure, the variant may have enhanced activity compared to wild-type or non-modified proteins, without being limited thereto.

As used herein, the term “conservative substitution” refers to substitution of one amino acid with a different amino acid having similar structural and/or chemical properties. The variant may have at least one conservative substitution while retaining at least one biological activity. Such amino acid substitution may generally occur based on similarity of polarity, charge, solubility, hydrophobicity, hydrophilicity, and/or amphipathic nature of a residue.

Variants may also include deletion or addition of amino acids that have minimal influence on properties and a secondary structure of a polypeptide. For example, the polypeptide may be conjugated to a signal (or leader) sequence at the N-terminus of a protein which co-translationally or post-translationally directs transfer of the protein. The polypeptide may also be conjugated with another sequence or linker to identify, purify, or synthesize the polypeptide.

In the present disclosure, “substituted with a different amino acid” is not particularly limited as long as the amino acid after substitution is different from the amino acid before substitution. That is, substitution of cysteine that is the 86th amino acid from the N-terminus of the amino acid sequence of SEQ ID NO: 1 with a different amino acid may also be expressed as “substitution of the 86th amino acid with an amino acid other than cysteine”. Meanwhile, in the present disclosure, it is obvious that the expression “substituted with a predetermined amino acid” means that the amino acid after substitution is different from the amino acid before substitution unless the expression “substituted with a different amino acid” is given.

The “glutamate-cysteine ligase variant” of the present disclosure may also be referred to as “a (variant) polypeptide having the glutamate-cysteine ligase activity” or “GSH1 variant” that may increase glutathione production compared to a protein before modification, a wild-type polypeptide, or a non-modified polypeptide, without being limited thereto.

In the variant, at least one amino acid of the amino acid sequence of SEQ ID NO: 1 may be substituted with a different amino acid. Specifically, the variant may include a substitution of the amino acid corresponding to the 86^{th} position of the amino acid sequence of SEQ ID NO: 1 with an amino acid other than cysteine. The different amino acid may be selected from glycine, alanine, valine, leucine, isoleucine, methionine, phenylalanine, tryptophan, proline, serine, threonine, tyrosine, asparagine, glutamate, glutamine, aspartate, lysine, arginine, and histidine.

In the present disclosure, it is obvious that the “variant in which the 86^{th} amino acid from the N-terminus of the amino acid sequence of SEQ ID NO: 1 is substituted with a different amino acid” includes a variant in which an amino acid corresponding to the 86^{th} position of the amino acid sequence of SEQ ID NO: 1 is substituted with a different amino acid, although the amino acid is at a position other than the 86^{th} position due to deletion/addition/addition or the like of an amino acid at the N- or C-terminus or in the middle of the amino acid sequence of SEQ ID NO: 1.

Also, although the variant, in which the 86^{th} amino acid from the N-terminus of the amino acid sequence of SEQ ID NO: 1 is substituted with a different amino acid, is disclosed as an example of the glutamate-cysteine ligase variant in the present disclosure, the glutamate-cysteine ligase of the present disclosure variant is not limited to the variant of the amino acid sequence of SEQ ID NO: 1, and it is obvious that a variant in which “the amino acid corresponding to the 86^{th} position of the amino acid sequence of SEQ ID NO: 1” is substituted with a different amino acid in any amino acid sequence having the activity of the glutamate-cysteine ligase is also within the scope of the glutamate-cysteine ligase variant of the present disclosure.

In any amino acid sequence, the “amino acid corresponding to the 86^{th} position of the amino acid sequence of SEQ ID NO: 1” may be identified by various sequence alignment methods well known in the art.

The glutamate-cysteine ligase variant of the present disclosure in which the amino acid corresponding to the 86^{th} position from the N-terminus of an amino acid sequence of SEQ ID NO: 1 is substituted with a different amino acid may be a protein including the amino acid sequence of SEQ ID NO: 1 or an amino acid sequence having at least 80%, 85%, 90%, 95%, 96%, 97%, 98%, or 99% homology or identity therewith in which an amino acid corresponding to the 86^{th} position of SEQ ID NO: 1 is substituted with a different amino acid.

The glutamate-cysteine ligase variant in which the 86^{th} amino acid from the N-terminus of the amino acid sequence of SEQ ID NO: 1 is substituted with an amino acid other than cysteine according to the present disclosure may include one of the amino acid sequences of SEQ ID NOS: 3 to 21. Specifically, the variant may consist essentially of one of the amino acid sequences of SEQ ID NOS: 3 to 21, and more specifically, it may consist of one of the amino acid sequences of SEQ ID NOS: 3 to 21, but is not limited thereto.

Also, the variant may include one of the amino acid sequences of SEQ ID NOS: 3 to 21 or an amino acid sequence including the fixed 86^{th} amino acid (i.e., in the amino acid sequence of the variant, the amino acid corresponding to the 86^{th} position of the SEQ ID NO: 3 to 21 is identical to the amino acid at the 86^{th} position of the SEQ ID NO: 3 to 21) and having at least 80% homology or identity therewith, but is not limited thereto.

Specifically, the variant of the present disclosure may include a polypeptide having one of the amino acid sequences of SEQ ID NOS: 3 to 21 and a polypeptide having at least 80%, 90%, 95%, 96%, 97%, 98%, or 99% homology or identity with one of the amino acid sequences of SEQ ID NOS: 3 to 21. Also, it is obvious that any protein having the amino acid sequence including a deletion, modification, substitution, or addition of one or several amino acids at a position other than the 86^{th} position is within the scope of the present disclosure as long as the protein retains the above-described homology or identity and an effect equivalent to that of the variant.

As used herein, the term “homology” or “identity” refers to a degree of relevance between two given amino acid sequences or base sequences and may be expressed as a percentage. The terms homology and identity may often be used interchangeably.

Sequence homology or identity of conserved polynucleotides or polypeptides may be determined by way of a standard alignment algorithm, and default gap penalties established by a program may be used together therewith. Substantially, homologous or identical sequences may hybridize with each other for at least about 50%, 60%, 70%, 80%, or 90% of the entire sequence or the entire length under moderate or highly stringent conditions. In hybridized polynucleotides, polynucleotides including a degenerate codon instead of a codon may also be considered.

The sequence homology, similarity, or identity between two given polypeptides or polynucleotides may be determined using any known computer algorithm such as the “FASTA” program by using default parameters as introduced by, for example, Pearson et al. (1988) Proc. Natl. Acad. Sci. USA 85:2444. Alternatively, the Needleman-Wunsch algorithm (1970, J. Mol. Biol. 48:443-453) performed in the Needleman program of The European Molecular Biology Open Software Suite (EMBOSS) package (Rice et al., 2000, Trends Genet. 16:276-277) (version 5.0.0 or later) may be used to determine the same (including the GCG program package (Devereux, J. et al., Nucleic Acids Research 12:387 (1984)), BLASTP, BLASTN, FASTA (Atschul, S. F. et al., J MOLEC BIOL 215:403 (1990); Guide to Huge Computers, Martin J. Bishop, ed., Academic Press, San Diego, 1994; and CARILLO et al. (1988) SIAM J Applied Math 48:1073). For example, the homology, similarity, or identity may be determined using BLAST, from the National Center for Biotechnology Information database, or ClustalW.

The homology, similarity, or identity between polynucleotides or polypeptides may be determined by comparing sequence information using the GAP computer program, such as a program introduced by Needleman et al. (1970), J Mol Biol. 48:443 as disclosed in Smith and Waterman, Adv. Appl. Math (1981) 2:482. In brief, the GAP program defines similarity as the number of aligned symbols (i.e., nucleotides or amino acids) which are similar, divided by the total number of symbols in the shorter of two sequences. Default parameters for the GAP program may include: (1) a binary comparison matrix (containing a value of 1 for identity and 0 for non-identity) a weighted comparison matrix of Gribskov et al. (1986) Nucl. Acids Res. 14:6745 disclosed in Schwartz and Dayhoff, eds., Atlas Of Protein Sequence And Structure, National Biomedical Research Foundation, pp. 353-358 (1979) (or EDNAFULL (EMBOSS version of NCBI NUC4.4) substitution matrix); (2) a penalty of 3.0 for each gap and an additional 0.10 penalty for each symbol in each gap (or gap open penalty of 10, and a gap extension penalty of 0.5); and (3) no penalty for end gaps.

Also, the sequence homology, similarity, or identity between two given polynucleotides or polypeptides may be identified by comparing sequences thereof by Southern hybridization under defined stringent conditions, and the defined stringent hybridization conditions are within the scope of the technology and may be defined by a method well known to one of ordinary skill in the art (For example, J. Sambrook et al., Molecular Cloning, A Laboratory Manual, 2nd Edition, Cold Spring Harbor Laboratory press, Cold Spring Harbor, New York, 1989; F. M. Ausubel et al., Current Protocols in Molecular Biology, John Wiley & Sons, Inc., New York).

Another aspect of the present disclosure provides a polynucleotide encoding the variant.

As used herein, the term “polynucleotide” refers to a polymer of nucleotides in which nucleotide monomers are linked to each other in a long chain shape by covalent bonds and generally means a DNA or RNA strand having a certain minimum length, more specifically a polynucleotide fragment encoding the variant.

The polynucleotide encoding the variant of the protein according to the present disclosure may include any nucleotide sequence encoding the glutamate-cysteine ligase variant having enhanced activity without limitation.

A gene encoding the glutamate-cysteine ligase of the present disclosure may be gsh1 gene. The gene may be derived from yeast. Specifically, the gene may be derived from a microorganism belonging to the genus Saccharomyces, more specifically Saccharomyces cerevisiae. Specifically, the gene may be a gene encoding the amino acid sequence of SEQ ID NO: 1, more specifically a sequence including a base sequence of SEQ ID NO: 2, but is not limited thereto.

The polynucleotide of the present disclosure may include various modifications made in a coding region provided not to change the amino acid sequence of the polypeptide expressed from the coding region by codon degeneracy or in consideration of codons preferred by a living organism in which the polypeptide is expressed. Specifically, any polynucleotide sequence encoding a protein variant in which the amino acids at a position corresponding to the 86^{th} position of SEQ ID NO: 1 is substituted with a different amino acid may be included therein without limitation. For example, the polynucleotide of the present disclosure may have a polynucleotide sequence encoding the protein variant of the present disclosure, specifically a protein including one of the amino acid sequences of SEQ ID NOS: 3 to 21 or a polypeptide having homology or identity therewith, without being limited thereto. The homology or identity is as described above.

In addition, the polynucleotide may include a nucleotide sequence that is hybridized with a probe constructed using a known gene sequence, e.g., a nucleotide sequence entirely or partly complementary to the nucleotide sequence under stringent conditions to encode the protein variant, in which the amino acid corresponding to the 86^{th} position of the amino acid sequence of SEQ ID NO: 1 is substituted with a different amino acid, without limitation.

The term “stringent conditions” refers to conditions which permit specific hybridization between polynucleotides. Such conditions are disclosed in detail in known documents (e.g., J. Sambrook et al.). For example, the conditions may include performing hybridization between genes having a high homology, e.g., a homology of 80% or more, specifically 90% or more, more specifically 95% or more, even more specifically 97% or more, and most specifically 99% or more, without performing hybridization between genes having a homology lower than the above homologies, or performing hybridization once, specifically two or three times, under conventional washing conditions for Southern hybridization at a salt concentration and temperature of 60° C., 1×SSC, and 0.1% SDS, specifically 60° ° C., 0.1×SSC, 0.1% SDS, and more specifically 68° C., 0.1×SSC, and 0.1% SDS. However, the stringent conditions are not limited thereto and may be appropriately adjusted by those skilled in the art according to the intended purposes.

Hybridization requires that two polynucleotides have complementary sequences, although mismatch of bases is possible according to the degree of stringency of hybridization. The term “complementary” is used to describe the relationship between nucleotide bases capable of hybridizing with each other. For example, with respect to DNA, adenosine is complementary to thymine, and cytosine is complementary to guanine. Thus, the present disclosure may include not only a substantially similar nucleic acid sequence but also an isolated nucleic acid fragment complementary to the entire sequence.

Specifically, the polynucleotides having homology or identity may be detected using the above-described hybridization conditions including a hybridization process at a Tₘ value of 55° C. Also, the Tₘ value may be, but is not limited to, 60° C., 63° C. or 65° C., and may be appropriately adjusted by those skilled in the art according to the intended purposes.

An appropriate degree of stringency for hybridization of polynucleotides may depend on lengths of the polynucleotides, and a degree of complementarity and parameters thereof are well known in the art.

Another aspect of the present disclosure provides a vector including a polynucleotide encoding the protein variant.

As used herein, the term “vector” refers to a DNA construct including a base sequence of a polynucleotide encoding a target protein, which is operably linked to an appropriate regulatory sequence to express the target protein in a suitable host cell. The regulatory sequence may include a promoter allowing initiating transcription, an operator sequence for regulating the transcription, a sequence encoding a suitable mRNA ribosome binding site, and a sequence regulating termination of transcription and translation. After the vector is introduced into the suitable host cell, it may replicate or function independently of the host genome and may be integrated into the genome itself.

The vector used in the present disclosure is not particularly limited, and any vector known in the art may be used. As a vector expressed by yeast, both integrative yeast plasmids (YIp) and extrachromosomal plasmid vectors may be used. The extrachromosomal plasmid vector may include episomal yeast plasmids (YEp), replicative yeast plasmids (YRp), and yeast centromer plasmids (YCp). Also, artificial yeast chromosomes (YACs) may also be used as the vector of the present disclosure. As a specific example, available vectors may include pESCHIS, pESC-LEU, pESC-TRP, pESC-URA, Gateway pYES-DEST52, pAO815, pGAPZ A, pGAPZ B, pGAPZ C, pGAPα A, pGAPα B, pGAPα C, pPIC3.5K, pPIC6 A, pPIC6 B, pPIC6 C, pPIC6α A, pPIC6α B, pPIC6α C, pPIC9K, pYC2/CT, PYD1 Yeast Display Vector, pYES2, pYES2/CT, pYES2/NT A, pYES2/NT B, pYES2/NT C, pYES2/CT, pYES2.1, pYES-DEST52, pTEF1/Zeo, pFLD1, PichiaPink™, p427-TEF, p417-CYC, pGAL-MF, p427-TEF, p417-CYC, PTEF-MF, pBY011, pSGP47, pSGP46, pSGP36, pSGP40, ZM552, pAG303GAL-ccdB, pAG414GAL-ccdB, pAS404, pBridge, pGAD-GH, pGAD T7, pGBK T7, pHIS-2, pOBD2, pRS408, pRS410, pRS418, pRS420, pRS428, yeast micron A form, pRS403, pRS404, pRS405, pRS406, pYJ403, pYJ404, pYJ405, and pYJ406, without being limited thereto.

For example, a polynucleotide encoding a target protein in the chromosome may be replaced by a mutated polynucleotide using a vector for chromosomal insertion into cells. The insertion of the polynucleotide into the chromosome may be performed by any method known in the art, for example, homologous recombination, without being limited thereto. A selection marker may be further included to confirm chromosomal insertion. The selection marker is used to select cells that are transformed with the vector, that is, to confirm insertion of a desired polynucleotide, and examples of the selection marker may include markers providing selectable phenotypes, such as drug resistance, nutrient requirement, resistance to cytotoxic agents, or surface expression of variant polypeptide. Only cells expressing the selection marker are able to survive or to show different phenotypes under the environment treated with a selective agent, and thus the transformed cells may be selected.

As used herein, the term “transformation” refers to a process of introducing the vector including the polynucleotide encoding the glutamate-cysteine ligase variant into a host cell in such a way that the protein encoded by the polynucleotide is expressed in the host cell. The transformed polynucleotide may be either in a form inserted into the chromosome of the host cell or in a form located outside the chromosome as long as the protein is expressed in the host cell. In addition, the polynucleotide includes DNA and RNA encoding the glutamate-cysteine ligase variant. The polynucleotide may be introduced into the host cell in any form as long as the polynucleotide is introduced into the host cell and the protein is expressed therein. For example, the polynucleotide may be introduced into the host cell in the form of an expression cassette that is a gene construct including all of the essential elements required for self-replication. The expression cassette may generally include a promoter operably linked to the polynucleotide, a transcription termination signal, a ribosome binding site, and a translation termination signal. The expression cassette may be in the form of a self-replicable expression vector. Also, the polynucleotide may be introduced into the host cell in its original form and operably linked to a sequence required for the expression in the host cell, without being limited thereto.

In addition, as used herein, the term “operably linked” means a functional linkage between a polynucleotide sequence encoding the polypeptide of the present disclosure and a promoter sequence which initiates and mediates transcription of the polynucleotide sequence. Methods for the transformation according to the present disclosure include any methods enabling introduction of the vector into the host cell, and may be performed by suitable standard techniques well known in the art selected according to the host cell. For example, electroporation, calcium phosphate (CaPO₄) precipitation, calcium chloride (CaCl₂)) precipitation, microinjection, a polyethylene glycol (PEG) method, a DEAE-dextran method, a cationic liposome method, and a lithium acetate-DMSO method may be used, but the present disclosure is not limited thereto.

The present disclosure may provide a microorganism producing glutathione by including at least one of: the variant; a polynucleotide encoding the variant; and a vector including the polynucleotide.

The microorganism may be a microorganism expressing the variant or a microorganism into which the variant is introduced.

As used herein, the term “microorganism including the variant”, “microorganism into which the variant is introduced”, or “microorganism expressing the variant” may refer to a microorganism prepared by enhancing the ability to produce glutathione to a microorganism naturally having a low ability to produce glutathione or by providing the ability to produce glutathione to a parent strain unable to produce glutathione. Specifically, the microorganism may be a microorganism expressing the glutamate-cysteine ligase variant including at least one amino acid mutation in the amino acid sequence of SEQ ID NO: 1, and the amino acid mutation may include a substitution of the amino acid corresponding to the 86^{th} position from the N-terminus with a different amino acid. In addition, the microorganism may be a microorganism expressing the glutamate-cysteine ligase variant in which the amino acid corresponding to the 86^{th} position of the amino acid sequence of SEQ ID NO: 1 is substituted with a different amino acid, without being limited thereto.

The glutamate-cysteine ligase and variants thereof are as described above.

As used herein, a protein “to be expressed/being expressed” means a state in which a target protein is introduced into or expressed in a microorganism. In the case where the protein is present in the microorganism, the activity of the protein is enhanced compared to the activity of an endogenous protein thereof or that before modification. In view of the objects of the present disclosure, the “target protein” may be the above-described glutamate-cysteine ligase variant.

Specifically, the term “introduction of a protein” refers to providing activity of a particular protein to a microorganism which does not possess the protein or enhancing the activity of the protein compared to the intrinsic activity of the protein or the activity before modification. For example, the introduction of a protein may refer to introduction of a polynucleotide encoding a particular protein into a chromosome or introduction of a fragment or vector including the polynucleotide encoding the particular protein into a microorganism, thereby expressing the activity of the protein. In addition, the “enhancement of activity” may mean that the activity of a particular protein of a microorganism is enhanced when compared with the intrinsic activity or the activity before modification. The term “intrinsic activity” refers to activity of a particular protein possessed by a parent strain before transformation when a microorganism is transformed by natural or artificial genetic variation.

Specifically, enhancement of the activity according to the present disclosure may be performed by at least one of the methods including increasing the copy number of a gene encoding the protein variant, introducing mutation into an expression regulatory sequence of the gene encoding the protein variant, replacing the expression regulatory sequence of the gene encoding the protein variant with a sequence having stronger activity, replacing a chromosomal gene encoding a wild-type protein with a gene encoding the protein variant, additionally introducing mutation into the gene encoding the protein variant in order to enhance the activity of the protein variant, and introducing the protein variant into a microorganism, without being limited thereto.

Although not particularly limited thereto, the increase in the copy number of the gene is performed in a form operably linked to a vector or in a form integrated into a chromosome of a host cell. Specifically, this method may be performed by introducing into a host cell a vector which replicates and functions irrespective of a host and is operably linked to a polynucleotide encoding the protein of the present disclosure. Alternatively, the copy number of the gene may be increased by introducing into a host cell a vector which inserts the polynucleotide into the chromosome of the host cell and is operably linked to the polynucleotide. The insertion of the polynucleotide into the chromosome may be performed by any method well known in the art, for example, homologous recombination.

Next, the modification of the expression regulatory sequence to increase the expression of the polynucleotide may be performed by inducing a variation in the nucleotide acid sequence by deletion, insertion, non-conservative substitution, conservative substitution, or any combination thereof to further enhance the activity of the expression regulatory sequence, or by replacing the nucleotide sequence with a nucleotide sequence having a stronger activity, without being limited thereto. The expression regulatory sequence may include a promoter, an operator sequence, a ribosome binding site encoding sequence, and a sequence for regulating transcription and translation, without being limited thereto.

A stronger promoter than the intrinsic promoter may be linked upstream of the polynucleotide expression unit, without being limited thereto. For example, when the host cell is yeast, the available promotor may include a TEF1 promoter, a TEF2 promoter, a GAL10 promoter, a GAL1 promoter, an ADH1 promoter, an ADH2 promoter, a PHO5 promoter, a GAL1-10 promoter, a TDH3 promoter (GPD promoter), a TDH2 promoter, a TDH1 promoter, a PGK1 promoter, a PYK2 promoter, an ENO1 promoter, an ENO2 promoter, and a TPI1 promoter, without being limited thereto. In addition, the modification of the polynucleotide sequence on the chromosome described may be performed by inducing a variation in the expression regulatory sequence by deletion, insertion, non-conservative substitution, conservative substitution, or any combination thereof to further enhance the activity of the polynucleotide sequence, or by replacing the nucleotide sequence with a nucleotide sequence modified to have a stronger activity, without being limited thereto.

Generally, the introduction and enhancement of the protein activity may increase the activity or concentration of the corresponding protein by 1%, 10%, 25%, 50%, 75%, 100%, 150%, 200%, 300%, 400%, or 500%, to a maximum of 1000% or 2000%, from the activity or concentration of a wild-type or non-modified microorganism strain, without being limited thereto.

As used herein, the term “non-modified microorganism” does not exclude strains having mutation that may occur naturally in microorganisms and may be a wild-type strain, a microorganism not including the protein variant, or a microorganism not transformed with a vector including a polynucleotide encoding the protein variant.

In the present disclosure, the microorganism including the glutamate-cysteine ligase variant or the polynucleotide encoding the same may be, for example, a recombinant microorganism prepared by transforming the microorganism with the vector including the polynucleotide, but is not limited thereto. The recombinant microorganism may be yeast, for example, a microorganism belonging to the genus Saccharomyces, specifically Saccharomyces cerevisiae. For example, the microorganism may be a Saccharomyces cerevisiae strain with Accession No. KCCM12659P, but is not limited thereto.

As used herein, the term “glutathione” may be interchangeably used with “GSH” and refers to a tripeptide compound consisting of three amino acids: glutamate, cysteine, and glycine. Glutathione may be used as a raw material for pharmaceuticals, health functional foods, taste ingredients, food and feed additives, cosmetics, and the like, but is not limited thereto.

As used herein, the term “glutathione-producing microorganism” includes microorganisms modified by natural or artificial genetic modification and may refer to a microorganism having a particular mechanism weakened or enhanced via introduction of an exogenous gene or enhancement or inactivation of an endogenous gene by genetic modification in order to produce glutathione. In view of the objects of the present disclosure, the microorganism producing glutathione may refer to a microorganism including the glutamate-cysteine ligase and capable of producing a large amount of target glutathione compared to wild-type or non-modified microorganisms. The “glutathione-producing microorganism” may be used interchangeably with “microorganism producing glutathione”, “microorganism having the ability to produce glutathione”, “glutathione-producing strain”, “strain having the ability to produce glutathione”, or the like.

The glutathione-producing microorganism may be a recombinant microorganism. The recombinant microorganism is as described above.

The type of the glutathione-producing microorganism is not particularly limited as long as glutathione is produced thereby, but may be a microorganism belonging to the genus Saccharomyces, specifically Saccharomyces cerevisiae, without being limited thereto.

A parent strain of the glutathione-producing microorganism including the variant is not particularly limited as long as the strain has the ability to produce glutathione. The microorganism may include modification for enhancement of a biosynthetic pathway for increasing glutathione producing ability, release of feedback inhibition, and inactivation of genes that weaken the degradation pathway or biosynthetic pathway, and such modification does not exclude naturally occurring modification. However, the present disclosure is not limited thereto.

Another aspect of the present disclosure provides a method of producing glutathione including culturing the microorganism. The microorganism and glutathione are as described above. Glutathione may be accumulated in the strain by culturing the strains.

With regard to a culture medium or other culturing conditions for culturing the strain of the present disclosure, any culture media commonly used to culture microorganisms belonging to the genus Saccharomyces may be used without limitation. Specifically, the strain of the present disclosure may be cultured in an ordinary medium containing appropriate carbon sources, nitrogen sources, phosphorus sources, inorganic compounds, amino acids, and/or vitamins under aerobic or anaerobic conditions while adjusting temperature, pH, and the like.

In the present disclosure, as the carbon sources, carbohydrates such as glucose, fructose, sucrose, and maltose; sugar alcohols such as mannitol and sorbitol; organic acids such as pyruvic acid, lactic acid, and citric acid; and amino acids such as glutamate, methionine, and lysine may be used, without being limited thereto. In addition, natural organic nutrients such as starch hydrolysates, molasses, blackstrap molasses, rice bran, cassava, sugar cane bagasse, and corn steep liquor may be used, and carbohydrates such as glucose and sterile pretreated molasses (i.e., molasses converted to reduced sugars) may be used, and suitable amounts of any other carbon sources may also be used without limitation. These carbon sources may be used alone or in a combination of at least two thereof.

As the nitrogen sources, inorganic nitrogen sources such as ammonia, ammonium sulfate, ammonium chloride, ammonium acetate, ammonium phosphate, ammonium carbonate, and ammonium nitrate; and organic nitrogen sources such as amino acids, peptone, NZ-amine, meat extract, yeast extract, malt extract, corn steep liquor, casein hydrolysate, fish or degradation products thereof, and defatted soybean cake or degradation products thereof may be used. These nitrogen sources may be used alone or in a combination of at least two thereof.

As the phosphorus sources, monopotassium phosphate, dipotassium phosphate, or sodium-containing salts corresponding thereto may be used. As the inorganic compounds, sodium chloride, calcium chloride, iron chloride, magnesium sulfate, iron sulfate, manganese sulfate, calcium carbonate, and the like may be used.

The culture medium may further include amino acids, vitamins, and/or suitable precursors. Specifically, L-amino acids or the like may be added to the culture medium for the strain. Specifically, glycine, glutamate, and/or cysteine may be added to the culture medium, and L-amino acids such as lysine may further be added thereto, if required, but the present disclosure is not limited thereto.

The culture medium and precursors may be added to cultures in a batch or continuous process, without being limited thereto.

In the present disclosure, during the culturing process of the strain, compounds such as ammonium hydroxide, potassium hydroxide, ammonia, phosphoric acid, and sulfuric acid may be properly added to the cultures in order to adjust the pH of the cultures. Also, a defoaming agent such as fatty acid polyglycol ester may be added in order to inhibit formation of foams during culturing. In addition, oxygen or an oxygen-containing gas may be injected into the cultures to maintain the cultures in an aerobic condition, and nitrogen, hydrogen, or carbon dioxide gases may be injected into the cultures to maintain the culture in anaerobic and micro-aerobic conditions without injecting any other gases therefor.

The temperature of the cultures may be maintained at 25° C. to 40° C., more specifically at 28° C. to 37° C., without being limited thereto. The culturing may be continued until a desired amount of a desired substance is obtained, specifically for 1 hour to 100 hours, without being limited thereto.

The method of producing glutathione may further include an additional process after the culturing step. The additional process may appropriately be selected in accordance with the purpose of use of glutathione.

Specifically, the method of producing glutathione may include recovering glutathione from at least one selected from the strain, a dried product, an extract, a culture product, and a lysate thereof after the culturing of the microorganism.

The method may further include lysing the strain before or simultaneously with the recovering step. The lysing of the strain may be performed by any method commonly used in the art to which the present disclosure pertains, e.g., by heat treatment or by using a buffer solution for lysis, a sonicator, and a French press. Also, the lysing step may include an enzymatic reaction by a cell wall lytic enzyme, nuclease, transnucleotidase, protease, or the like, without being limited thereto.

In view of the objects of the present disclosure, dry yeast, yeast extract, yeast extract mix powder, and pure glutathione, each having a high glutathione content, may be prepared by way of the method of producing glutathione. However, the present disclosure is not limited thereto, and these products may be appropriately prepared according to desired products.

In the present disclosure, the dry yeast may be used interchangeably with “dried product of the strain”. The dry yeast may be prepared by drying the yeast strain in which glutathione is accumulated, and specifically, it may be included in a feed composition, a food composition, and the like, without being limited thereto.

In the present disclosure, the yeast extract may be used interchangeably with terms such as “extract of the strain”. The extract of the strain may refer to substances remaining after separating cell walls from the strains. Specifically, the extract of the strain may refer to remaining components, excluding cell walls, among components obtained by lysing the cells. The extract of the strain includes glutathione and one or more other components selected from proteins, carbohydrates, nucleic acids, and fibers in addition to glutathione, without being limited thereto.

The recovering step may be performed using any suitable method well known in the art, and glutathione, as the target substance, may be recovered.

The recovering step may include a purification process. The purification process may be performed by isolating only glutathione from the strain. Via the purification process, pure glutathione may be prepared.

If required, the method of preparing glutathione may further include mixing an excipient with one selected from the strain, a dried product, an extract, a culture, and a lysate thereof, and glutathione recovered therefrom. By way of the mixing step, a yeast extract mix powder may be prepared.

The excipient may be appropriately selected and used according to the intended use or form and may be, for example, selected from starch, glucose, cellulose, lactose, glycogen, D-mannitol, sorbitol, lactitol, maltodextrin, calcium carbonate, synthetic aluminum silicate, calcium monohydrogen phosphate, calcium sulfate, sodium chloride, sodium hydrogen carbonate, purified lanolin, dextrin, sodium alginate, methyl cellulose, colloidal silica gel, hydroxypropyl starch, hydroxypropylmethyl cellulose, propylene glycol, casein, calcium lactate, primojel, and Arabia gum, and specifically, it may include at least one component selected from starch, glucose, cellulose, lactose, dextrin, glycogen, D-mannitol, and maltodextrin, without being limited thereto.

The excipient may include, for example, a preservative, a humectant, a dispersant, a suspending agent, a buffer, a stabilizer, or an isotonic agent, without being limited thereto.

Another aspect of the present disclosure provides a use of the glutamate-cysteine ligase variant for producing glutathione. Another aspect of the present disclosure provides a use of the microorganism for producing glutathione. The glutamate-cysteine ligase variant, the microorganism, and glutathione are as described above.

### MODE FOR INVENTION

Hereinafter, the present disclosure will be described in more detail with reference to the following examples and experimental examples. However, the following examples and experimental examples are merely presented to exemplify the present invention, and the scope of the present invention is not limited thereto.

### Example 1: Selection of Glutathione-Producing Strain and Confirmation of Glutathione Producing Ability

Strains were obtained from yeast blocks containing various strains, and characteristics thereof were improved to select strains having a glutathione producing ability.

Specifically, grain samples such as rice, barley, mung beans, and oats were collected from 20 areas, such as, Hwaseong, Pyeongtaek, Yongin, and the like of Gyeonggi-do, Republic of Korea, pulverized, kneaded, wrapped in a cloth, pressed firmly to form a shape, wrapped with straw for fermentation for 10 days, and slowly dried to prepare yeast blocks.

The following experiment was performed to isolate various strains from the prepared yeast blocks. 45 mL of a saline solution was added to 5 g of yeast blocks and pulverized using a mixer. For purely isolating yeast strains, the resultant was diluted by serial dilution, spread on YPD agar (10 g/L yeast extract, 20 g/L bacto peptone, and 20 g/L glucose per 1 L of distilled water), and cultured at 30° ° C. for 48 hours. Then, according to the morphology of colonies and microscopic verification, colonies of the yeast were streaked on the YPD agar. 25 mL of YPD broth was seeded in a 250 ml conical flask, and the purely isolated strain was inoculated thereonto and cultured in a shaking incubator for 48 hours at 30° C. and 200 rpm. Strains were screened by identifying glutathione production.

In order to improve primarily isolated strains, random mutation was induced in the isolated strains. Specifically, a strain confirmed to have the glutathione producing ability was isolated from the yeast blocks and designated as CJ-37 strain. The CJ-37 strain was cultured in a solid medium and inoculated into a broth to obtain a culture solution thereof, and the culture solution was exposed to UV light using a UV lamp. After the culture solution exposed to UV rays was plated on a plate medium, only a mutated strain that had formed colonies was isolated, and glutathione production thereof was identified.

As a result, among the mutated strains, a strain exhibiting the greatest glutathione production was selected as a glutathione-producing strain, named CJ-5 strain, deposited at the Korean Culture Center of Microorganisms (KCCM) under the Budapest Treaty, and designated as Accession No. KCCM12568P on Jul. 31, 2019.

### Example 2: Experiment for Further Improving Glutathione Producing Ability

Mutation was induced in the following manner in order to further improve the glutathione producing ability of the CJ-5 strain.

The CJ-5 strain was cultured in a solid medium and inoculated into a broth to obtain a culture solution, and the culture solution was exposed to UV light using a UV lamp. After the culture solution exposed to UV light was plated on a plate medium, only a mutated strain that had formed colonies was isolated. A strain exhibiting the greatest glutathione production was isolated, named CC02-2490 strain, deposited at the Korean Culture Center of Microorganisms (KCCM) under the Budapest Treaty, and designated as Accession No. KCCM12659P on Jan. 17, 2020. As a result of analyzing the base sequence of the gsh1, which is a glutathione biosynthesis gene, with respect to enhancement of the glutathione producing ability of the strain, it was confirmed that cysteine, which is the 86^{th} amino acid of the GSH1 protein encoded by the gsh1 gene, had been substituted with arginine.

### Example 3: Experiment for Mutation of GSH1 C86 Residue

Considering that the amino acid at the 86^{th} position of the of the GSH1 protein is important in glutathione production based on the results of Example 2, mutant strains of Saccharomyces cerevisiae (S. cerevisiae) CEN.PK2-1D and Saccharomyces cerevisiae (S. cerevisiae) CJ-5 were prepared such that protein variants were expressed in which cysteine at the 86^{th} position of the GSH1 protein was substituted with a different amino acid, and increases in glutathione production were identified.

In order to prepare strains in which cysteine at the 86^{th} position of the GSH1 protein of Saccharomyces cerevisiae was substituted with arginine, pWAL100 and pWBR100 plasmids were used with reference to a thesis of Lee T H et al. (J. Microbiol. Biotechnol. (2006), 16(6), 979-982). Specifically, polymerase chain reaction (PCR) was performed using genomic DNA of the CJ-5 strain as a template as follows. A partial sequence of the N-terminus of the GSH1 protein including a N-terminal BamHI flanking sequence, a GSH1 ORF initiation codon, and a C86R mutation-encoding sequence was obtained by performing PCR using primers of SEQ ID NOS: 22 and 23, and a partial sequence of the C-terminus of the GSH1 protein including a C-terminal XhoI flanking sequence, a GSH1 ORF termination codon, and a C86R mutation-encoding sequence was obtained by performing PCR using primers of SEQ ID NOS: 24 and 25. Subsequently, as a result of performing overlap PCR using the two sequences as templates with the primers of SEQ ID NOS: 22 and 25, a GSH1 ORF fragment, including a sequence encoding a GSH1-modified protein was obtained in which cysteine at the 86^{th} position was substituted with arginine and N-terminal BamHI and C-terminal XhoI restriction enzyme sequences. The ORF fragment was treated with BamHI and XhoI and then cloned into a pWAL100 vector treated with the same enzymes to prepare a pWAL100-GSH1(C86R) vector.

Also, the 500 bp downstream from the GSH1 ORF termination codon including N-terminal SpeI and C-terminal NcoI restriction enzyme sequences was obtained by performing PCR using the genomic DNA of the CJ-5 strain as a template and primers of SEQ ID NOS: 26 and 27 and treated with SpeI and NcoI restriction enzymes. Subsequently, the resultant was cloned into pWBR100 treated with the same enzymes to prepare a pWBR100-GSH1 vector.

For preparing a final DNA fragment to be introduced into the yeast, PCR products including the sequence encoding arginine mutation and a part of KIURA3 were obtained using the pWAL100-GSH1(C86R) vector prepared as described above as a template and the primers of SEQ ID NOS: 22 and 28, and PCR products including a part of KIURA3 and the 500 bp downstream from the GSH1 termination codon were obtained using the pWBR100-GSH1 vector as a template and the primers of SEQ ID NOS: 29 and 27. S. cerevisiae CEN.PK2-1D and S. cerevisiae CJ-5 were transformed with the PCR products in the same molar ratio. PCR was performed via denaturation at 95° C. for 5 minutes, annealing at 53ºC for 1 minute, and polymerization for 1 minute per kb at 72° C., and the transformation of the yeast was carried out according to a lithium acetate method modified from a method disclosed in a thesis of Geitz (Nucleic Acid Research, 20(6), 1425). Specifically, yeast cells with an OD of 0.7 to 1.2 were washed with a lithium acetate/TE buffer twice and mixed with the PCR products and single-stranded DNA (Sigma D-7656). The cells were cultured under a static culture condition in a lithium acetate/TE/40% PEG buffer at 30° C. for 30 minutes and at 42ºC for 15 minutes. Then, the cells cultured in a SC (2% glucose) agar plate not including uracil until colonies were visible to obtain a strain into which the GSH1 C86R mutation-encoding sequence and the KIURA3 gene were introduced. Subsequently, in order to remove KIURA3, the strains were cultured in 2 mL of YPD overnight, diluted at a ratio of 1/100, plated on an SC (2% glucose) agar plate including 0.1% 5-FOA to prepare S. cerevisiae CEN.PK2-1D GSH1 C86R mutated strain and S. cerevisiae CJ-5 GSH1 C86R mutated strain from which a uracil marker had been removed. Strains capable of expressing GSH1-modified proteins in which cysteine was substituted with 18 types of amino acids other than arginine were also prepared in the same manner, except that a primer pair of SEQ ID NOS: 23 and 24 was used in which the sequence encoding the 86^{th} arginine was substituted with a sequence encoding a different amino acid.

TABLE 1

| Primer | 5′→3′ sequence |
|---|---|
| F_BamHI_GSH1 | GGTAGGATCCATGGGACTCTTAGCTTTGGGCAC |
| (SEQ ID | |
| NO: 22) | |
| R_GSH1_C86R | TTAGCCTCCCTAAGGGACGAATCCT |
| (SEQ ID | |
| NO: 23) | |
| F_GSH1_C86R | CGTCCCTTAGGGAGGCTAACGATGT |
| (SEQ ID | |
| NO: 24) | |
| R_XhoI_GSH1 | ATGACTCGAGTTAACATTTGCTTTCTATTGAAGGC |
| (SEQ ID | |
| NO: 25) | |
| F_SpeI_ | TAGAACTAGTACTCCTTTTATTTCGGTTGTGAA |
| GSH1_DW | |
| (SEQ ID | |
| NO: 26) | |
| R_NcoI_ | GCTGCCATGGGAATAGTGTGAACCGATAACTGTGT |
| GSH1_DW | |
| (SEQ ID | |
| NO: 27) | |
| R_AL | GAGCAATGAACCCAATAACGAAATCTT |
| killer | |
| (SEQ ID | |
| NO: 28) | |
| F_BR | CTTGACGTTCGTTCGACTGATGAG |
| killer | |
| (SEQ ID | |
| NO: 29) | |

After culturing the strains prepared as described above for 26 hours, concentrations of produced glutathione (GSH) were measured and listed in Tables 2 and 3.

TABLE 2
S. cerevisiae CEN.PK2-1D

| | GSH concentration | |
|---|---|---|
| | (mg/L) | |
| Mutant | 26 hr | Increase(fold) |
| WT | 86.0 | 1.00 |
| F | 109.5 | 1.27 |
| H | 103.2 | 1.20 |
| K | 100.7 | 1.17 |
| E | 100.1 | 1.16 |
| G | 99.5 | 1.16 |
| D | 97.5 | 1.13 |
| N | 96.3 | 1.12 |
| R | 95.2 | 1.11 |
| Y | 94.6 | 1.10 |
| I | 93.0 | 1.08 |
| L | 92.2 | 1.07 |
| P | 92.0 | 1.07 |
| W | 90.8 | 1.06 |
| Q | 90.3 | 1.05 |
| S | 90.0 | 1.05 |
| M | 89.9 | 1.05 |
| T | 86.6 | 1.01 |
| A | 86.4 | 1.00 |
| V | 86.2 | 1.00 |

TABLE 3
S. cerevisiae CJ-5

| | GSH concentration | |
|---|---|---|
| | (mg/L) | |
| Mutant | 26 hr | Increase(fold) |
| WT | 271.3 | 1.00 |
| R | 330.0 | 1.22 |
| N | 321.9 | 1.19 |
| D | 318.4 | 1.17 |
| E | 314.4 | 1.16 |
| P | 304.2 | 1.12 |
| K | 302.4 | 1.11 |
| A | 294.9 | 1.09 |
| Q | 286.4 | 1.06 |
| V | 285.6 | 1.05 |
| F | 282.5 | 1.04 |
| Y | 277.5 | 1.02 |
| W | 276.0 | 1.02 |
| S | 274.5 | 1.01 |
| T | 273.9 | 1.01 |
| I | 273.5 | 1.01 |
| H | 272.1 | 1.00 |
| G | 272.0 | 1.00 |

Based on the results of the experiment, it was confirmed that the glutathione producing ability obtained by substituting cysteine at the 86^{th} position of the GSH1 protein with a different amino acid increased up to 27% compared to the glutathione producing ability obtained by the wild-type GSH1 protein.

Based thereon, it was confirmed that the GSH1 variant prepared by substituting cysteine at the 86^{th} position of the GSH1 protein with a different amino acid had significantly improved glutathione producing ability.

### Example 4: Confirmation of Effect of Another Cys Residue on Glutathione Production

As a comparative example, another Cys residue of the GSH1 protein was modified, and the glutathione producing ability obtained by the modification was identified. The results are shown in Table 4 below.

TABLE 4
S. cerevisiae CJ-5

| | GSH concentration | |
|---|---|---|
| | (mg/L) | |
| Mutant | 26 hr | Increase(fold) |
| WT | 267.7 | 1.00 |
| C70A | 255.7 | 0.96 |
| C164A | 208.2 | 0.78 |
| C213A | 228.2 | 0.85 |
| C264A | 238.9 | 0.89 |
| C266A | 174.2 | 0.65 |
| C571A | 234.5 | 0.88 |
| C642A | 254.6 | 0.95 |
| C678A | 250.4 | 0.94 |

Referring to the results of the experiment, a cysteine residue other than the cysteine at the 86^{th} position had no effects on increasing glutathione production, but instead decreased the glutathione production.

Based thereon, it was confirmed that not all cysteine residues present in the protein have effects on increasing glutathione production. Also, it was confirmed that the novel GSH1 variant developed in the present disclosure increased glutathione production.

Since the yeast producing glutathione with a high yield, a dried product, an extract, a culture, and a lysate thereof, and the produced glutathione have antioxidant, detoxification, and immunity-enhancing effects, they may be efficiently used to prepare the cosmetic composition, the food composition, the feed composition, and the pharmaceutical composition.

The above description of the present invention is provided for the purpose of illustration, and it would be understood by those skilled in the art that various changes and modifications may be made without changing the technical conception and essential features of the present invention. Thus, it is clear that the above-described embodiments are illustrative in all aspects and do not limit the present invention. The various embodiments disclosed herein are not intended to be limiting, with the true scope and spirit being indicated by the following claims. The present invention is to be limited only by the terms of the appended claims, along with the full scope of equivalents to which such claims are entitled.

1. A glutamate-cysteine ligase variant in which an amino acid corresponding to the 86^{th} position from the N-terminus of an amino acid sequence of SEQ ID NO: 1 is substituted with a different amino acid.

2. The glutamate-cysteine ligase variant of claim 1, wherein the amino acid corresponding to the 86^{th} position is substituted with glycine, alanine, valine, leucine, isoleucine, methionine, phenylalanine, tryptophan, proline, serine, threonine, tyrosine, asparagine, glutamate, glutamine, aspartate, lysine, arginine, or histidine.

3. The glutamate-cysteine ligase variant of claim 1, wherein the variant has a sequence homology equal to or greater than 80% and less than 100% with the amino acid sequence of SEQ ID NO: 1.

4. The glutamate-cysteine ligase variant of claim 1, wherein the variant consists of one amino acid sequence selected from SEQ ID NOS: 3 to 21.

5. A polynucleotide encoding the glutamate-cysteine ligase variant according to claim 1.

6. A vector comprising the polynucleotide of claim 5.

7. A microorganism producing glutathione comprising at least one of: the glutamate-cysteine ligase variant according to claim 1; a polynucleotide encoding the variant; and a vector comprising the polynucleotide.

8. The microorganism of claim 7, wherein the microorganism is a microorganism belonging to the genus Saccharomyces.

9. The microorganism of claim 7, wherein the microorganism is Saccharomyces cerevisiae.

10. The microorganism of claim 7, wherein the microorganism is a Saccharomyces cerevisiae strain deposited with Accession No. KCCM12659P.

11. A method of producing glutathione, the method comprising culturing a microorganism comprising at least one of: the glutamate-cysteine ligase variant according to claim 1; a polynucleotide encoding the variant; and a vector comprising the polynucleotide, in a culture medium.

12. The method of claim 11, further comprising recovering glutathione from at least one selected from the cultured microorganism, a dried product of the microorganism, an extract of the microorganism, a culture of the microorganism, and a lysate of the microorganism.


SEQUENCE LISTING (29 sequences)

SEQ ID NO: 1 - protein, 678 aa, Saccharomyces cerevisiae
MGLLALGTPLQWFESRTYNEHIRDEGIEQLLYIFQAAGKRDNDPLFWGDELEYMVVDFDDKERNSMLDVCHDKILTELNMEDSSLCEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 2 - DNA, 2037 bp, Saccharomyces cerevisiae
atgggactcttagctttgggcacgcctttgcagtggtttgagtctaggacgtacaatgaacacataagggatgaaggtatcgagcagttgttgtatattttccaagctgctggtaaaagagacaatgaccctcttttttggggagacgagcttgagtacatggttgtagattttgatgataaggagagaaattctatgctcgacgtttgccatgacaagatactcactgagcttaatatggaggattcgtccctttgtgaggctaacgatgtgagttttcaccctgagtatggccggtatatgttagaggcaacaccagcttctccatatttgaattacgtgggtagttacgttgaggttaacatgcaaaaaagacgtgccattgcagaatataagctatctgaatatgcgagacaagatagtaaaaataacttgcatgtgggctccaggtctgtccctttgacgctgactgtcttcccgaggatgggatgccccgactttattaacattaaggatccgtggaatcataaaaatgccgcttccaggtctctgtttttacccgatgaagtcattaacagacatgtcaggtttcctaacttgacagcatccatcaggaccaggcgtggtgaaaaagtttgcatgaatgttcccatgtataaagatatagctactccagaaacggatgactccatctacgatcgagattggtttttaccagaagacaaagaggcgaaactggcttccaaaccgggtttcatttatatggattccatgggttttggcatgggctgttcgtgcttacaagtgacctttcaggcacccaatatcaacaaggcacgttacctgtacgatgcattagtgaattttgcacctataatgctagccttctctgccgctgcgcctgcttttaaaggttggctagccgaccaagatgttcgttggaatgtgatatctggtgcggtggacgaccgtactccgaaggaaagaggtgttgcgccattactacccaaatacaacaagaacggatttggaggcattgccaaagacgtacaagataaagtccttgaaataccaaagtcaagatatagttcggttgatcttttcttgggtgggtcgaaatttttcaataggacttataacgacacaaatgtacctattaatgaaaaagtattaggacgactactagagaatgataaggcgccactggactatgatcttgctaaacattttgcgcatctctacataagagatccagtatctacattcgaagaactgttgaatcaggacaacaaaacgtcttcaaatcactttgaaaacatccaaagtacaaattggcagacattacgttttaaaccccccacacaacaagcaaccccggacaaaaaggattctcctggttggagagtggaattcagaccatttgaagtgcaactattagattttgagaacgctgcgtattccgtgctcatatacttgattgtcgatagcattttgaccttttccgataatattaacgcatatattcatatgtccaaagtatgggaaaatatgaagatagcccatcacagagatgctatcctatttgaaaaatttcattggaaaaaatcatttcgcaacgacaccgatgtggaaactgaagattattctataagcgagattttccataatccagagaatggtatatttcctcaatttgttacgccaatcctatgccaaaaagggtttgtaaccaaagattggaaagaattaaagcattcttccaaacacgagagactatactattatttaaagctaatttctgatagagcaagcggtgaattgccaacaacagcaaaattctttagaaattttgtactacaacatccagattacaaacatgattcaaaaatttcaaagtcgatcaattatgatttgctttctacgtgtgatagacttacccatttagacgattcaaaaggtgaattgacatcctttttaggagctgaaattgcagaatatgtaaaaaaaaataagccttcaatagaaagcaaatgttaa

SEQ ID NO: 3 - protein, 678 aa, Artificial Sequence
  S.cerevisiae GSH1 C86A
MGLLALGTPLQWFESRTYNEHIRDEGIEQLLYIFQAAGKRDNDPLFWGDELEYMVVDFDDKERNSMLDVCHDKILTELNMEDSSLAEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 4 - protein, 678 aa, Artificial Sequence
  S.cerevisiae GSH1 C86D
MGLLALGTPLQWFESRTYNEHIRDEGIEQLLYIFQAAGKRDNDPLFWGDELEYMVVDFDDKERNSMLDVCHDKILTELNMEDSSLDEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 5 - protein, 678 aa, Artificial Sequence
  S.cerevisiae GSH1 C86E
MGLLALGTPLQWFESRTYNEHIRDEGIEQLLYIFQAAGKRDNDPLFWGDELEYMVVDFDDKERNSMLDVCHDKILTELNMEDSSLEEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 6 - protein, 678 aa, Artificial Sequence
  S.cerevisiae GSH1 C86F
MGLLALGTPLQWFESRTYNEHIRDEGIEQLLYIFQAAGKRDNDPLFWGDELEYMVVDFDDKERNSMLDVCHDKILTELNMEDSSLFEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 7 - protein, 678 aa, Artificial Sequence
  S.cerevisiae GSH1 C86G
MGLLALGTPLQWFESRTYNEHIRDEGIEQLLYIFQAAGKRDNDPLFWGDELEYMVVDFDDKERNSMLDVCHDKILTELNMEDSSLGEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 8 - protein, 678 aa, Artificial Sequence
  S.cerevisiae GSH1 C86H
MGLLALGTPLQWFESRTYNEHIRDEGIEQLLYIFQAAGKRDNDPLFWGDELEYMVVDFDDKERNSMLDVCHDKILTELNMEDSSLHEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 9 - protein, 678 aa, Artificial Sequence
  S.cerevisiae GSH1 C86I
MGLLALGTPLQWFESRTYNEHIRDEGIEQLLYIFQAAGKRDNDPLFWGDELEYMVVDFDDKERNSMLDVCHDKILTELNMEDSSLIEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 10 - protein, 678 aa, Artificial Sequence
  S.cerevisiae GSH1 C86K
MGLLALGTPLQWFESRTYNEHIRDEGIEQLLYIFQAAGKRDNDPLFWGDELEYMVVDFDDKERNSMLDVCHDKILTELNMEDSSLKEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 11 - protein, 678 aa, Artificial Sequence
  S.cerevisiae GSH1 C86L
MGLLALGTPLQWFESRTYNEHIRDEGIEQLLYIFQAAGKRDNDPLFWGDELEYMVVDFDDKERNSMLDVCHDKILTELNMEDSSLLEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 12 - protein, 678 aa, Artificial Sequence
  S.cerevisiae GSH1 C86M
MGLLALGTPLQWFESRTYNEHIRDEGIEQLLYIFQAAGKRDNDPLFWGDELEYMVVDFDDKERNSMLDVCHDKILTELNMEDSSLMEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 13 - protein, 678 aa, Artificial Sequence
  S.cerevisiae GSH1 C86N
MGLLALGTPLQWFESRTYNEHIRDEGIEQLLYIFQAAGKRDNDPLFWGDELEYMVVDFDDKERNSMLDVCHDKILTELNMEDSSLNEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 14 - protein, 678 aa, Artificial Sequence
  S.cerevisiae GSH1 C86P
MGLLALGTPLQWFESRTYNEHIRDEGIEQLLYIFQAAGKRDNDPLFWGDELEYMVVDFDDKERNSMLDVCHDKILTELNMEDSSLPEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 15 - protein, 678 aa, Artificial Sequence
  S.cerevisiae GSH1 C86Q
MGLLALGTPLQWFESRTYNEHIRDEGIEQLLYIFQAAGKRDNDPLFWGDELEYMVVDFDDKERNSMLDVCHDKILTELNMEDSSLQEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 16 - protein, 678 aa, Artificial Sequence
  S.cerevisiae GSH1 C86R
MGLLALGTPLQWFESRTYNEHIRDEGIEQLLYIFQAAGKRDNDPLFWGDELEYMVVDFDDKERNSMLDVCHDKILTELNMEDSSLREANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 17 - protein, 678 aa, Artificial Sequence
  S.cerevisiae GSH1 C86S
MGLLALGTPLQWFESRTYNEHIRDEGIEQLLYIFQAAGKRDNDPLFWGDELEYMVVDFDDKERNSMLDVCHDKILTELNMEDSSLSEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 18 - protein, 678 aa, Artificial Sequence
  S.cerevisiae GSH1 C86T
MGLLALGTPLQWFESRTYNEHIRDEGIEQLLYIFQAAGKRDNDPLFWGDELEYMVVDFDDKERNSMLDVCHDKILTELNMEDSSLTEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 19 - protein, 678 aa, Artificial Sequence
  S.cerevisiae GSH1 C86V
MGLLALGTPLQWFESRTYNEHIRDEGIEQLLYIFQAAGKRDNDPLFWGDELEYMVVDFDDKERNSMLDVCHDKILTELNMEDSSLVEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 20 - protein, 678 aa, Artificial Sequence
  S.cerevisiae GSH1 C86W
MGLLALGTPLQWFESRTYNEHIRDEGIEQLLYIFQAAGKRDNDPLFWGDELEYMVVDFDDKERNSMLDVCHDKILTELNMEDSSLWEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 21 - protein, 678 aa, Artificial Sequence
  S.cerevisiae GSH1 C86Y
MGLLALGTPLQWFESRTYNEHIRDEGIEQLLYIFQAAGKRDNDPLFWGDELEYMVVDFDDKERNSMLDVCHDKILTELNMEDSSLYEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 22 - DNA, 33 bp, Artificial Sequence
  F_BamHI_GSH1
ggtaggatccatgggactcttagctttgggcac

SEQ ID NO: 23 - DNA, 25 bp, Artificial Sequence
  R_GSH1_C86R
ttagcctccctaagggacgaatcct

SEQ ID NO: 24 - DNA, 25 bp, Artificial Sequence
  F_GSH1_C86R
cgtcccttagggaggctaacgatgt

SEQ ID NO: 25 - DNA, 35 bp, Artificial Sequence
  R_XhoI_GSH1
atgactcgagttaacatttgctttctattgaaggc

SEQ ID NO: 26 - DNA, 33 bp, Artificial Sequence
  F_SpeI_GSH1_DW
tagaactagtactccttttatttcggttgtgaa

SEQ ID NO: 27 - DNA, 35 bp, Artificial Sequence
  R_NcoI_GSH1_DW
gctgccatgggaatagtgtgaaccgataactgtgt

SEQ ID NO: 28 - DNA, 27 bp, Artificial Sequence
  R_AL killer
gagcaatgaacccaataacgaaatctt

SEQ ID NO: 29 - DNA, 24 bp, Artificial Sequence
  F_BR killer
cttgacgttcgttcgactgatgag